(19) United States
(12) Patent Application Publication
(10) Pub. No.: US 2024/0226439 A9
(43) Pub. Date: Jul. 11, 2024

(54) BLOW-FILL SEALING METHOD FOR FILLING AND PACKAGING

(71) Applicant: Merck Sharp & Dohme LLC, Rahway, NJ (US)

(72) Inventors: Carl Chew, Swedesboro, NJ (US); Matthew H. Flamm, Ambler, PA (US); Jeremy A. Haddock, Wilson, NC (US); Rikhabh Jain, Belle Mead, NJ (US); Atul Karande, Lansdale, PA (US); Brit L. Rudeen, Bethlehem, PA (US)

(73) Assignee: Merck Sharp & Dohme LLC, Rahway, NJ (US)

(21) Appl. No.: 18/484,485

(22) Filed: Oct. 11, 2023

Related U.S. Application Data
(60) Provisional application No. 63/418,067, filed on Oct. 21, 2022.

(51) Int. Cl. A61M 5/24; A61J 1/06; B29C 49/04; B29C 49/42; B65B 3/02; B65D 1/09

(52) U.S. Cl. CPC A61M 5/2455; A61J 1/067; B29C 49/04; B29C 49/42808; B65B 3/022; B65D 1/095; A61M 2207/10

(57) ABSTRACT

A method of controlling a temperature of a biopharmaceutical product during blow-fill sealing, includes developing a model based on heat transfer mechanisms that incorporates the effects of the blow-fill sealing on the biopharmaceutical product, providing a parison to a blow-fill sealing machine for accepting the biopharmaceutical product during the blow-fill sealing, predicting a temperature of at least one the biopharmaceutical product, the parison, and a component of the blow-fill sealing machine at a stage in the blow-fill sealing, and adjusting a parameter of the blow-fill sealing machine to reduce damage to the biopharmaceutical product.

### CROSS-REFERENCE TO RELATED APPLICATIONS

This application claims the benefit of priority to U.S. Provisional Patent Application No. 63/418,067, filed Oct. 21, 2022.

### FIELD OF THE DISCLOSURE

The present disclosure relates to methods for forming pharmaceutical containers and for modeling the manufacturing process during blow-fill sealing. More particularly the present disclosure relates to methods of forming and modeling such containers with precise temperature controls.

### BACKGROUND OF THE DISCLOSURE

Glass vials are commonly used to store vaccines, biologics, medicaments, and the like. Syringes are used to extract the contents of the vials and administer the medicament to the patient. Pre-filled syringes may also be used. Such syringes typically utilize parenteral routes (e.g., injecting directly into the body, bypassing the skin and mucous membranes), and are administered by a physician or healthcare provider. Recently, automatic injection devices (commonly known as “autoinjectors”) have been used to simplify the administration of drugs in certain settings. Due to their simplicity of design, autoinjectors allow patients to use the devices on themselves reliably and safely, and in their own home without supervision. Certain patients may experience pain or fear of needles while using conventional syringes and autoinjectors, especially in multi-dosage treatments, and recurring therapeutic applications. Additionally, conventional devices may not be able to allow for proper sustained drug release, and may not be suitable for patients of all ages.

To address this, ampoules are being developed to store and deliver certain biopharmaceutical products (e.g., vaccines, gene therapy, etc.). The products stored in ampoules are often temperature-sensitive and at risk of degradation during the ampoule formation and/or filling process. This degradation may result, for example, in a loss of potency or a reduced shelf-life. Thus, there exists a need for methods and devices that improve upon and advance the methods of forming and filling these ampoules.

### SUMMARY OF THE DISCLOSURE

The disclosure describes systems and methods for controlling a temperature of a biopharmaceutical product during blow-fill sealing. In certain aspects, a method of controlling a temperature of a biopharmaceutical product during blow-fill sealing is provided which includes: providing a parison to a blow-fill sealing machine for accepting the biopharmaceutical product during the blow-fill sealing; and applying at least one heat transfer mechanism to control the temperature for the biopharmaceutical product.

In some embodiments, the heat transfer mechanism is selected from: (i) heat transfer from a pre-molded parison and air inside a parison balloon; (ii) heat transfer between a molded parison and the main mold before, during and/or after introduction of the biopharmaceutical product; (iii) heat transfer between the molded parison and a mold; (iv) heat transfer between the molded parison and the biopharmaceutical product during and/or after introduction of the biopharmaceutical product; (v) heat transfer between a fully formed ampoule after molding and an external environment; (vi) heat transfer between an ampoule and the biopharmaceutical product during inversion of the ampoule; or (vii) continued heat transfer between the ampoule and the external environment.

In some embodiments, at least two, at least three, at least four, at least five, or at least six of heat transfer mechanisms (i) through (vii) are applied to control the temperature for the biopharmaceutical product. In some embodiments, heat transfer mechanisms (i) through (vii) are applied.

In some embodiments, the method of controlling a temperature of a biopharmaceutical product during blow-fill sealing further comprises cooling a component of the blow-fill sealing machine to ensure that a temperature of the biopharmaceutical product does not exceed a predetermined threshold. In some embodiments, the predetermined threshold is selected based on a sensitivity of the biopharmaceutical product.

In some embodiments, the predetermined threshold is 20° C., 25° C., 30° C., or 35° C. In certain embodiments, the predetermined threshold is 35° C.

In some embodiments, the method of controlling a temperature of a biopharmaceutical product during blow-fill sealing further comprises decreasing a temperature of a mold of the blow-fill sealing machine.

In some embodiments, the method of controlling a temperature of a biopharmaceutical product during blow-fill sealing further comprises decreasing a temperature of a mandrel of the blow-fill sealing machine. In certain embodiments, the mandrel is maintained at a temperature below 15° C.

In certain aspects, a system for controlling a temperature of a biopharmaceutical product during blow-fill sealing is provided which includes: a blow-fill sealing machine including a mandrel configured to introduce the biopharmaceutical product, and a mold to receive a ribbon of parison and form one or more ampoules; and at least one cooling subunit configured and arranged to reduce a temperature of the biopharmaceutical product.

In some embodiments, the at least one cooling subunit includes a mandrel cooling subunit that recirculates a substance through an outer annular space of the mandrel.

In some embodiments, the at least one cooling subunit includes a mold cooling subunit including a standalone temperature control unit that supplies a substance to the mold. In some embodiments, the substance is propylene glycol.

In some embodiments, the at least on cooling subunit includes one or more air knives configured to direct a stream of air onto the ribbon of parison. In some embodiments, the one or more air knives includes a pair of 12″ air knives placed on opposing ends of the ribbon and configured to blow air onto the ribbon and away from the mandrel.

In certain aspects, a method of controlling a temperature of a biopharmaceutical product during blow-fill sealing is provided which includes: developing a model based on heat transfer mechanisms that incorporates effects of the blow-fill sealing on the biopharmaceutical product; providing a parison to a blow-fill sealing machine for accepting the biopharmaceutical product during the blow-fill sealing; predicting a temperature of at least one of the biopharmaceutical product, the parison, and a component of the blow-fill sealing machine at a stage in the blow-fill sealing; and adjusting a parameter of the blow-fill sealing machine to reduce damage to the biopharmaceutical product.

In some embodiments, predicting a temperature includes predicting the biopharmaceutical product temperature during a filling process. In some embodiments, predicting a temperature includes predicting the biopharmaceutical product temperature after a filling process. In some embodiments, predicting a temperature includes predicting a parison temperature during a filling process. In some embodiments, predicting a temperature includes predicting a mandrel or mold temperature during a filling process.

In some embodiments, developing a model comprises incorporating at least one of parison thermal properties, a parison thickness, product properties, an inlet temperature, and temperature setpoints for a mold, a mandrel, a buffer tank, and a product piping.

In some embodiments, developing a model comprises incorporating at least one of seven calculations including: (i) heat transfer from a pre-molded parison and air inside a parison balloon; (ii) heat transfer between a molded parison and the main mold before, during and/or after introduction of the biopharmaceutical product; (iii) heat transfer between the molded parison and a mold; (iv) heat transfer between the molded parison and the biopharmaceutical product during and/or after introduction of the biopharmaceutical product; (v) heat transfer between a fully formed ampoule after molding and an external environment; (vi) heat transfer between an ampoule and the biopharmaceutical product during inversion of the ampoule; and (vii) continued heat transfer between the ampoule and the external environment. In some embodiments, developing a model comprises incorporating all seven calculations.

In some embodiments, adjusting a parameter of the blow-fill sealing machine includes cooling a component of the blow-fill sealing machine to ensure that a temperature of the biopharmaceutical product does not exceed a predetermined threshold. In some embodiments, the predetermined threshold is selected based on a sensitivity of the biopharmaceutical product. In certain embodiments, the predetermined threshold is 35° C.

In some embodiments, adjusting a parameter of the blow-fill sealing machine includes decreasing a temperature of a mold of the blow-fill sealing machine.

In some embodiments, adjusting a parameter of the blow-fill sealing machine includes decreasing a temperature of a mandrel of the blow-fill sealing machine. In certain embodiments, decreasing a temperature of a mandrel includes keeping the mandrel at a temperature below 15° C.

In certain aspects, a system for controlling a temperature of a biopharmaceutical product during blow-fill sealing is provided which includes: a blow-fill sealing machine including a mandrel configured to introduce the biopharmaceutical product and a mold to receive a ribbon of parison and form one or more ampoules; and at least one cooling subunit configured and arranged to reduce a temperature of the biopharmaceutical product.

In some embodiments, the at least one cooling subunit includes a mandrel cooling subunit that recirculates a substance through an outer annular space of the mandrel. In some embodiments, the at least one cooling subunit includes a mold cooling subunit including a standalone temperature control unit that supplies a substance to the mold. In some embodiments, the substance is propylene glycol.

In some embodiments, the at least on cooling subunit includes one or more air knives configured to direct a stream of air onto the ribbon of parison. In some embodiments, the one or more air knives includes a pair of 12″ air knives placed on opposing ends of the ribbon and configured to blow air onto the ribbon and away from the mandrel.

In certain aspects, a method of controlling a temperature of a plastic during blow-fill sealing is provided which comprises: developing a model based on heat transfer mechanisms that incorporates effects of the blow-fill sealing on the biopharmaceutical product; providing a parison to a blow-fill sealing machine for accepting the biopharmaceutical product during the blow-fill sealing; predicting a temperature of at least one the biopharmaceutical product, the parison, and a component of the blow-fill sealing machine at a stage in the blow-fill sealing; and adjusting a parameter of the blow-fill sealing machine to produce the plastic with predetermined characteristics.

### BRIEF DESCRIPTION OF THE DISCLOSURE

Various embodiments of the presently disclosed ampoules are disclosed herein with reference to the drawings, wherein:

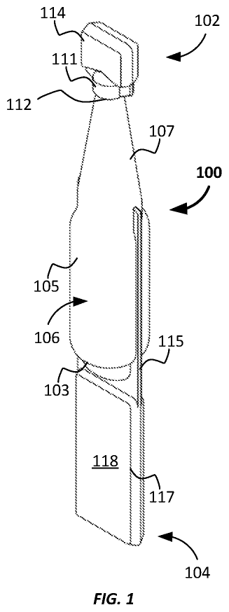

FIG. 1 is a schematic perspective view of a first embodiment of an ampoule;

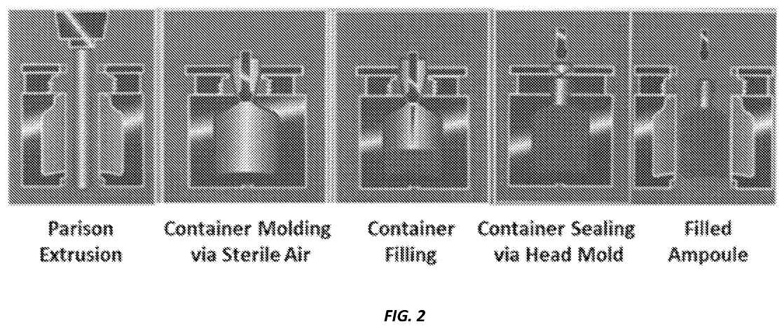

FIG. 2 illustrates a method of forming an ampoule using blow-fill sealing;

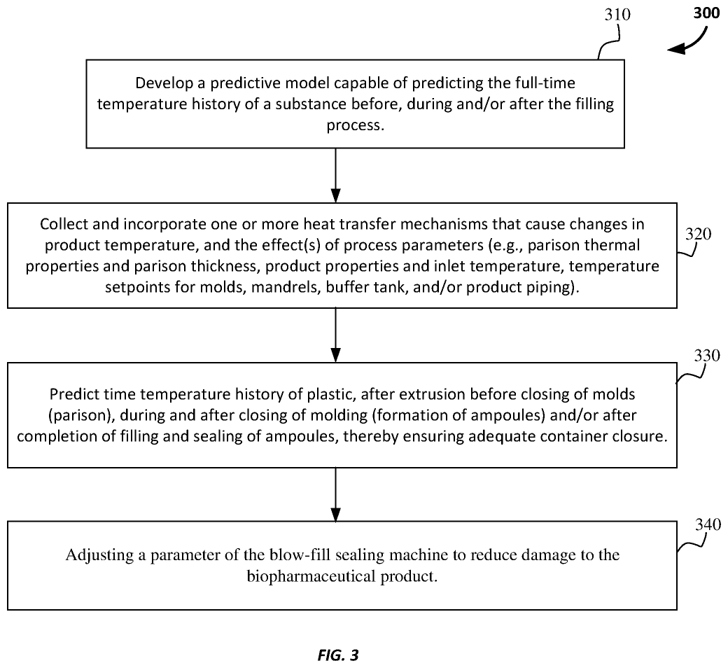

FIG. 3 is a schematic diagram showing the steps of a method for predicting time temperature history of plastic;

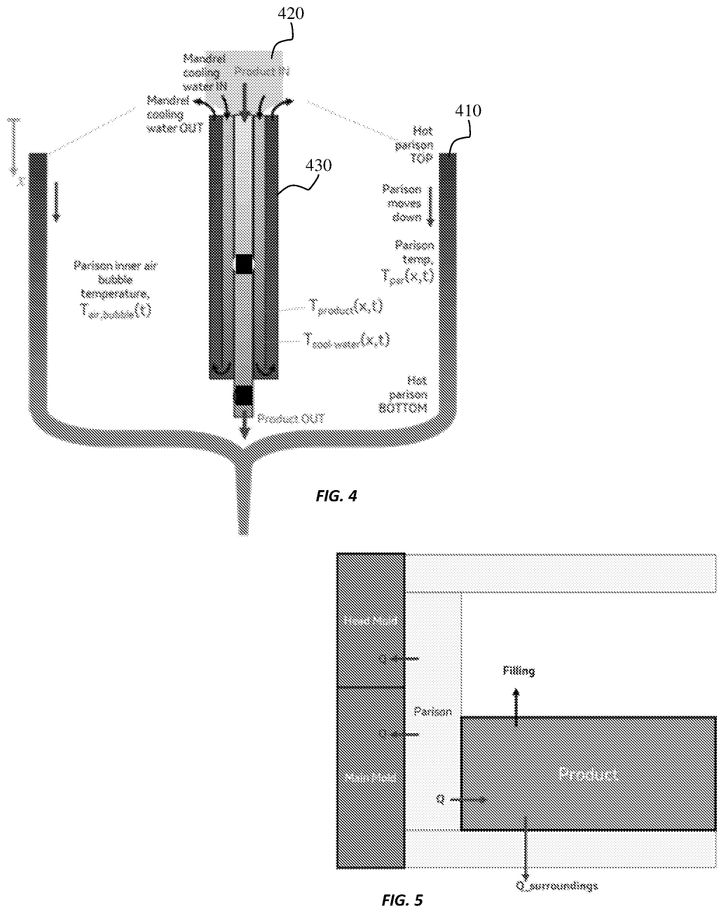

FIG. 4 is a schematic diagram showing a model of heat transfer between pre-molded parison and a biopharmaceutical product in a mandrel;

FIG. 5 is a schematic diagram showing a model of heat transfer during molding an introduction of a biopharmaceutical product; and

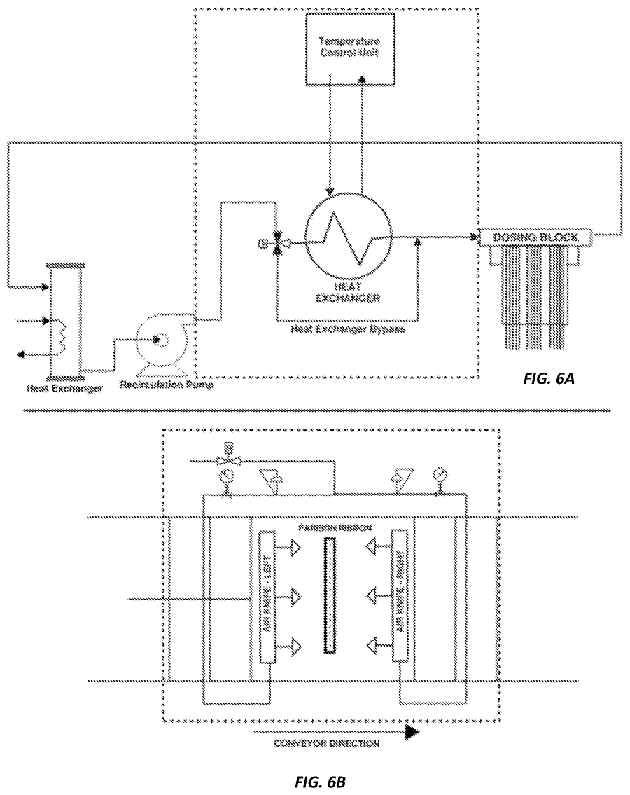

FIG. 6A is a schematic diagram of a system for blow-fill sealing having a heat exchanger bypass and a temperature control unit disposed between a recirculation pump and a dosing block. FIG. 6B is a schematic diagram of a system for blow-fill sealing having one or more air knives installed directly downstream of molds.

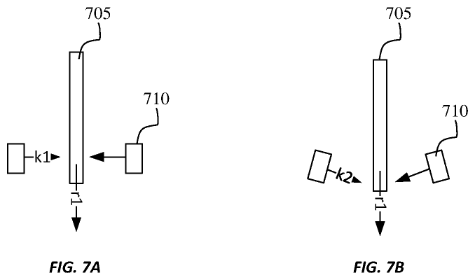

FIG. 7A is a schematic diagram of a system for blow-fill sealing having air knives positioned to blow compressed air in a direction of vector k1 that is perpendicular to the vertical movement r1 of a ribbon. FIG. 7B is a schematic diagram of a system for blow-fill sealing having air knives oriented or configured to blow compressed air in a direction of vector k2 that includes a vertical component in the same direction as the vertical movement r1 of a ribbon; and

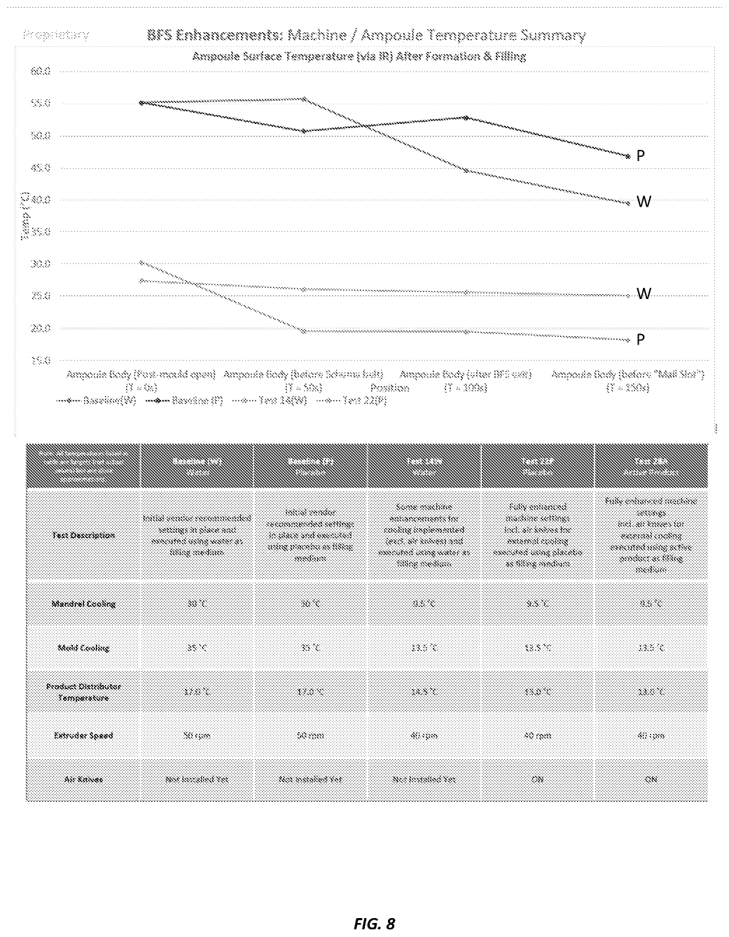

FIG. 8 shows a comparison between ampule surface temperature using several cooling techniques described herein.

Various embodiments of the present invention will now be described with reference to the appended drawings. It is to be appreciated that these drawings depict only some embodiments which may or may not all be required for functionality of the invention and are therefore not to be considered limiting of its scope.

### DETAILED DESCRIPTION

Despite the various improvements that have been made to drug delivery, conventional devices suffer from some shortcomings as described above.

Therefore, there is a need for further improvements to the devices, systems, and methods of delivering medicaments. Among other advantages, the present disclosure may address one or more of these critical needs.

As used herein, the term “proximal,” when used in connection with a component of an ampoule, refers to the end of the component closest to the delivery target (e.g., the patient's mouth when the ampoule is being administered to a patient), whereas the term “distal,” when used in connection with a component of an ampoule, refers to the end of the component farthest from the delivery target (e.g., patient's mouth during delivery). Likewise, the terms “trailing” and “leading” are to be taken as relative to the delivery target of the ampoule. “Trailing” is to be understood as relatively farther away from the delivery target, and “leading” is to be understood as relatively closer to the delivery target. As used herein, the terms “medicament,” “medication,” and “drug” are generally used interchangeably and it will be understood that the ampoules described herein may be used to store, deliver, or administer vaccines, biologics, therapeutics, medicaments, topical ointments, and the like.

As shown in FIG. 1, a first embodiment of an ampoule 100 will be described. Ampoule 100 may extend between a proximal end 102 and a distal end 104, and may be entirely formed of a polymer. In some examples, ampoule 100 may include polyolifins, such as Low-Density Polyethylene (LDPE) material, which can be flexible and able to administer a medicament when squeezed. In some examples, ampoule 100 is made of a translucent material (i.e., one that allows light to at least partially pass therethrough) that allows the user to at least partially see its contents. In some examples, the ampoule may have a predetermined opacity. In specific embodiments the ampoule has a predetermined opacity of 90%, 80%, 70%, 60%, 50%, 40%, 30%, 20%, or 10%. The methods of forming ampoule 100 will be described in greater detail below. Generally, ampoule 100 may include a base 103, a body 105 defining an inner cavity 106, a tapered neck 107 connected to the body and leading to a cap 111, the cap fitted on nozzle 112. In at least some examples, nozzle 112 may be disposed at the interface between the bottom of cap 111 and the top of neck 107. The inner diameter of nozzle 112 may be chosen based on a desired fluid flow speed. In at least some examples, the nozzle 112 may have a diameter from 1 mm to 4 mm. Nozzle 112 may form a predetermined percentage of the total surface area of the top of neck 107 (e.g., nozzle may form less than 2%, 2-5%, 5-10%, 10-20%, 20-30%, or more than 30% of the surface area of the top of neck 107). Cap 111 may be removably coupleable to nozzle 112. In at least some examples, cap 111 may be scored or define a weakened portion at its base so that the cap may be twisted and separated from the rest of the ampoule to expose nozzle 112.

Medicament may be disposed inside cavity 106 of body 105, which may define a continuous passage through neck 107 and out of nozzle 112. As shown in FIG. 1, cap 111 may be coupled to an anti-choking mechanism in the form of a miter 114, the miter having a width that is greater than the width of cap 111. In some examples, miter 114 is generally rectangular. Alternatively, miter 114 may be circular, oval, or butterfly-shaped. Miter 114 may be 13-15 mm in width and 10-15 mm in length. Miter 114 may be 25%, 30%, 40%, 50%, 75%, 100% or more wider than cap 111. In at least some examples, miter 114 is sized so that it will not pose a choking hazard to an infant. Cap 111 and miter 114 may be coupled together, or unitarily formed, so that the cap and miter remain together when the cap 111 is removed from the rest of the ampoule to expose the nozzle 112.

Optionally, the ampoule may include one or more flattened shoulders 115 coupled to, and extending around portions of, body 105. Shoulders 115 may also be coupled to platform 117. Platform 117 may be rectangular as shown, or circular, triangular, or any other desired shape. In at least some examples, platform 117 includes a flattened surface 118 capable of accepting an adhesive label that includes text or symbols to signify the contents of the ampoule, dosing instructions, adverse side effects, manufacturer, lot number, or other identifying or patient-specific information.

To administer medication, a user (e.g., patient or operator) may remove cap 111 from the ampoule 100 to expose the nozzle 112 by twisting, bending, or otherwise tearing the cap from neck 107. The user may then place the nozzle inside the patient's mouth and squeeze the flexible walls of the body together adjacent the cavity to eject the contents of the ampoule cavity into the mouth if oral dosing is intended. In some other examples, the medication may be ejected onto another body part as indicated by the medication. Thus, though delivery to the mouth is being primarily described, other delivery routes are also possible including nasal, rectal, or vaginal administrations, administrations to the eye (i.e., drops) or the ear, etc. These delivery routes are also applicable to all embodiments of an ampoule described in this disclosure. In embodiments where the miter 114 remains attached to cap 111, the ampoule may be used with infants and children without posing a choking risk from the separated cap. The neck and specifically the taper of the neck may have a length and/or width determined based on an intended delivery route (e.g., infant cheek) and the angle of taper may be chosen based on the delivery route. For example, neck 107 may have a length greater than 15 mm (e.g., 25 mm, 26 mm, 27 mm, 28 mm, 29 mm, 30 mm or greater), and a gradually narrowing width that is linearly, or non-linearly reduced from 15 mm at its base (i.e., closest to body 105) to 5 mm or less at its crown closest to cap 111. Overall, the shape of the neck may be substantially conical, and the neck may be angled with respect to the longitudinal axis at a neck angle α of between 5 degrees to 20 degrees (e.g., between 5 and 10 degrees, or at an angle of 8 degrees).

In some examples, the ampules described herein may be mass produced using blow-fill sealing (BFS). A schematic showing some of the steps of blow-fill sealing technology is shown in FIG. 2. Briefly, blow-fill sealing is a pharmaceutical filling process in which the primary packaging container is formed from an extruded thermoplastic parison, generally provided as a continuous ribbon of melted resin. The parison may be filled with product, and sealed in a continuous, integrated, automated operation. It has been referred to as an “advanced aseptic fill-seal technique” by the United States Food and Drug Administration (FDA) and United States Pharmacopeia (USP) due to its minimal human intervention. One advantage of blow-fill sealing over “traditional” aseptic filling operations is that personnel are not normally present in the filling area, thereby removing a potential source of microbial contamination. Instead, containers are formed immediately before filling, filled under controlled conditions, and sealed immediately after filling. The blow-fill sealing machine output may be dependent upon product and polymer (resin) physical characteristics and container design, and the cycle time may be dependent upon the product filling characteristics (e.g., viscosity, foaming) and the resin-dependent formation time required in the mold. As noted above, current blow-fill sealing technology may result in temperatures that are higher than desirable, and the elevated temperatures may damage the product inside the filled ampoules.

To address this, methods are presented for modeling, controlling, predicting, tracking, altering, and/or modulating the temperature(s) of one or more components of the blow-filling sealing machine, the parison, the formed ampoule, and/or the product contained therein. In some embodiments, a method of predicting and/or controlling the temperature of a biopharmaceutical product and/or ampoule is disclosed. Specifically, this method may relate to filling and/or packaging biopharmaceutical products with varying degrees of temperature sensitivities and/or thermal properties. In a first embodiment shown in FIG. 3, a method 300 provides the ability to predict and/or control the internal temperature of a biopharmaceutical product before, during, and/or after filling of the biopharmaceutical product into polymer ampoules. Prior state of the art consists of obtaining measurements of the internal product temperature and external ampoule-surface temperatures after filling, without any knowledge of actual product temperature history throughout the entire process. Conversely, method 300 includes developing a predictive model 310 that is capable of predicting the full time-temperature history during the entire filling process (e.g., prior to filling, during filling and/or after filling). In some examples, the temperature history of the biopharmaceutical product may be graphed or mapped over time during the process. This predictive model or “digital twin” may be trained by incorporating or integrating the thermal properties of the materials involved (e.g., the parison, the biopharmaceutical product, components of the BFS machine, etc.), obtained from literature and measurements. The model may also take into account one or more heat transfer mechanisms 320 that cause changes in product temperature, and the effect(s) of process input parameters including parison thermal properties and parison thickness, product properties and inlet temperature, temperature setpoints for molds, mandrels, buffer tank, and/or product piping, as well as other parameters. In some examples, predictive model 310 and data collected from 320 may also enable secondary prediction 330 of time temperature history of plastic, after extrusion but before closing of molds (parison), during and/or after closing of molding (e.g., during or after formation of ampoules), and/or after completion of filling and sealing of ampoules, thereby ensuring adequate container closure. The method may also include an additional step of adjusting a parameter of the blow-fill sealing machine (e.g., to cool a mandrel, to cool a mold, to add air knives, to increase a wall thickness of an ampoule, etc.) to reduce damage to the biopharmaceutical product 340.

FIG. 4 is a schematic representation of one model of heat transfer between a pre-molded parison 410 and the biopharmaceutical product 420 being introduced through mandrel 430. The product distributor is part of the product path located above the filling mandrels. As product 420 enters the distributor, flow is divided into each mandrel 420 for filling into ampoules.

Additionally, FIG. 5 provides a schematic of a model of heat transfer during molding and introduction of the product including heat transfer between the parison and the head mold and main mold, heat transfer between the parison and the product, and heat transfer between the product and the surroundings. The term “main mold” refers to the pair of mold halves that close on the parison to form the body of the ampoule, and the term “head mold” refers to the pair of mold halves that close on the parison after the ampoule body has been formed and filled to seal the ampoule. As a high-level description of the model attributes, any one or more of the following heat transfer mechanisms that affect the temperature of the biopharmaceutical product stream may be captured:
(1) Heat transfer from the hot pre-molded parison, and hot air inside parison balloon;
(2) Heat transfer between the molded parison and the main mold before, during, and after introduction of biopharmaceutical product;
(3) Separately, the heat transfer between the molded parison and the head mold;
(4) Heat transfer between the molded parison and the biopharmaceutical product during and/or after introduction of the biopharmaceutical product;
(5) Heat transfer between the fully formed ampoule after molding and the external environment with and without air knives;
(6) Heat transfer between ampoule and biopharmaceutical product during inversion of the ampoule; and
(7) Continued heat transfer between ampoule and external environment.

It will be understood that any one or more of the seven mechanisms, or equations or calculations representing the seven mechanisms, described above may be incorporated into the modeling. In one example, all seven mechanisms, or equations or calculations representing the seven mechanisms, may be incorporated into the model of heat transfer to estimate, model, predict, or control the temperature of a biopharmaceutical product and/or an ampoule. Additionally, it will be understood that these mechanisms are non-exhaustive and that other mechanisms may be incorporated into a model.

In a second embodiment, a method provides the ability to identify relevant operating parameters with a high degree of accuracy that may have an impact on minimizing biopharmaceutical product temperature exposure without impacting the quality of an ampoule. These parameters may include, for example, the temperature of a mandrel, the temperature of a mold, the wall thickness of an ampoule or the temperature of a ribbon. This embodiment may reduce the need for running extensive empirical experiments and trials on the blow-fill sealing machine using actual biopharmaceutical products. As the example illustrated in FIG. 4 shows, for a biopharmaceutical product with temperature sensitivity of 35° C. or below, the most influential parameter may be the temperature of product as it travels through the mandrels. Control of this parameter may be achieved by ensuring the mandrels temperature is set at a temperature lower than 15° C. Thus, the model described above may be correlated with a practical application of adjusting a parameter of the blow-fill sealing machine to reduce damage to the biopharmaceutical product. Adjusting a parameter of the blow-fill sealing machine may include cooling a component of the blow-fill sealing machine to ensure that a temperature of the biopharmaceutical product does not exceed a predetermined threshold. The predetermined threshold may be selected based on a sensitivity of the biopharmaceutical product. For example, the predetermined threshold may be 35° C. Adjusting a parameter of the blow-fill sealing machine may include decreasing a temperature of a mold and/or mandrel of the blow-fill sealing machine.

In a third embodiment, the devices and methods described herein may be used to control the temperature of a biopharmaceutical product before, during, and/or after filling of the biopharmaceuticals without compromising on the ability of a blow-fill sealing system to form ampoules of acceptable quality and/or without compromising the manufacturing capacity. For example, conventional techniques mainly focus on relatively slower blow-fill sealing shuttle-type machines and attempt to slow the machine throughput to achieve temperature control. However, the methods, devices, and techniques described herein may be applied to the relatively faster blow-fill sealing rotary-type machines without having to slow them down (i.e., the methods do not impact the throughput, defined as the number of containers processed per unit of time) while achieving the desired reduction in temperature exposure.

In some examples, the present disclosure contemplates a blow-fill sealing system having an internal cooling system for biopharmaceutical product piping, buffer tank, mandrels, and/or molds. Additionally, or alternatively, one or more modifications may be used to control biopharmaceutical product temperature. Generally, the cooling techniques described herein may include one or more of the following: systems for reduced operating temperature via mandrel cooling and/or mold cooling, additional temperature control unit(s) for increased cooling capacity at mandrels and/or molds, decreased extruder speed for lower ampoule wall thickness, for example, to reduce thermal heat transfer to product, and/or using one or more air knives for external cooling of an ampoule after molding and/or filling for reduced thermal heat transfer to a biopharmaceutical produce.

These systems and techniques may, for example, include a higher-capacity cooling system. In some embodiments, the cooling system includes a subsystem for mandrel cooling. The mandrel cooling subsystem may include an open to atmosphere reservoir filled with propylene glycol (e.g., 30% v/v) that utilizes a centrifugal pump to recirculate the cooling substance through the outer annular space of each mandrel. It will be understood that other cooling substances are also possible including chilled water, ethylene glycol, or combinations thereof. Temperature exchange within this reservoir may be controlled by manually adjusting a thermostatic valve to vary flow rate through a small ring of coils submerged within the reservoir, and the coil ring may use the existing main chiller Temperature Control Unit (TCU) set point temperature. In an alternative configuration, a standalone TCU and heat exchanger may be used to deliver additional cooling capacity to the existing mandrel cooling circuit. The standalone TCU may provide a nominal 10 kW (˜34,000 BTU/h) cooling capacity at 10° C. (return temperature) and the new plate and frame heat exchanger may have an effective surface area of 10.7 ft². It has been observed that this higher-capacity cooling system reduced biopharmaceutical product temperature by ˜27° C. as demonstrated during machine trials.

The cooling system may instead, or in addition, include a mold cooling subsystem. In one embodiment, a system includes a standalone TCU that supplied propylene glycol (30% v/v) to the molds. The standard TCU was not capable of achieving desired temperatures below 20° C. due to design limitation of the TCU. In another embodiment, this TCU was subsequently bypassed and provided direct-cooling from the Main Chiller TCU which enhanced the mold cooling ability down to −13° C. (return temperature). A schematic representation of the heat exchanger bypass and the use of the Temperature Control Unit disposed between the recirculation pump and the dosing block is shown in FIG. 6A.

Additionally, or alternatively, the cooling system may include a subsystem for external cooling of formed ampoules. In one embodiment, one or more air knives may be installed directly downstream of the molds, and a constant or intermittent stream of clean compressed air may be supplied to each air knife as shown in FIG. 6B. In some examples, two 12″ air knives may be disposed on either side of a ribbon of parison. Each air knife may use a standard 0.002″ shim and may be positioned approximately 6″ from the vertically descending ribbon. In some examples, two air knives are disposed equidistant from, or on opposing ends of, the ribbon. In some examples, the air knives 710 may be positioned to blow compressed air in a direction of vector k1 that is perpendicular to the vertical movement r1 of the ribbon 705 (i.e., the vector k1 may include a vertical component of 0) (FIG. 7A). Alternatively, the air knives 710 may be oriented or configured to blow compressed air in a direction of vector k2 that includes a vertical component in the same direction as the vertical movement r1 of the ribbon 705 (FIG. 7B). Stated another way, the vertical knives may blow compressed air away from the mold and/or mandrel. The air knives may blow air in a continuous fashion or may blow air intermittently or periodically as desired. This external cooling installation may include pressure monitoring instrumentation to allow development of process controls for the specific biopharmaceutical product and ampoule configuration. Without being bound by any particular theory, it is believed that air knives 710 may provide an additional amount (e.g., between 7° C. and 10° C.) of cooling of an ampoule surface and an additional amount (e.g., between 3° C. and 4° C.) of cooling of a biopharmaceutical product after ampoule formation during machine trials. Additionally, a biopharmaceutical product temperature reduction may be achieved from other cooling system modifications described above. Further enhancements can possibly be made by installing additional air knives in series and/or in parallel, using higher-capacity air knives, and/or other similar air-cooling methods (e.g., vortex cooling).

FIG. 8 illustrates the effects of the disclosed systems including mandrel cooling, mold cooling, product distributor temperature, extruder speed, and air knives. As shown, these techniques and methods are capable of producing a significant temperature reduction in the system, which may reduce the temperature of the biopharmaceutical product and reduce the risk of loss of potency. For example, baseline experiments using initial vendor recommended settings (i.e., 30° C. mandrel cooling, 35° C. mold cooling, 17° C. product distributor temperature, 50 rpm extruder speed, and no air knives) resulted in a final output temperature range of 45-55° C. for placebo (P) and water (W) samples, respectively. When fully enhanced using the methods and techniques described herein (i.e., 9° C. mandrel cooling, 13.5° C. mold cooling, 13° C. product distributor temperature, 40 rpm extruder speed, with knives), the final output temperature was decreased to less than 20° C. for a placebo sample (Test 22A). Similar results were achieved with an active product sample (Test 28A) using the fulling enhanced machine settings with air knives.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

It will be appreciated that the various dependent claims and the features set forth therein can be combined in different ways than presented in the initial claims. It will also be appreciated that the features described in connection with individual embodiments may be shared with other embodiments of the described embodiments.

What is claimed is:

1. A method of controlling a temperature of a biopharmaceutical product during blow-fill sealing, comprising:
providing a parison to a blow-fill sealing machine for accepting the biopharmaceutical product during the blow-fill sealing; and
applying at least one heat transfer mechanism to control the temperature for the biopharmaceutical product.

2. The method of claim 1, wherein the heat transfer mechanism is selected from: (i) heat transfer from a pre-molded parison and air inside a parison balloon; (ii) heat transfer between a molded parison and the main mold before, during and/or after introduction of the biopharmaceutical product; (iii) heat transfer between the molded parison and a mold; (iv) heat transfer between the molded parison and the biopharmaceutical product during and/or after introduction of the biopharmaceutical product; (v) heat transfer between a fully formed ampoule after molding and an external environment; (vi) heat transfer between an ampoule and the biopharmaceutical product during inversion of the ampoule; or (vii) continued heat transfer between the ampoule and the external environment.

3. The method of claim 2, wherein heat transfer mechanisms (i) through (vii) are applied.

4. The method of claim 1, further comprising cooling a component of the blow-fill sealing machine to ensure that a temperature of the biopharmaceutical product does not exceed a predetermined threshold.

5. The method of claim 4, wherein the predetermined threshold is selected based on a sensitivity of the biopharmaceutical product.

6. The method of claim 4, wherein the predetermined threshold is 35° C.

7. The method of claim 1, further comprising decreasing a temperature of a mold of the blow-fill sealing machine.

8. The method claim 1, further comprising decreasing a temperature of a mandrel of the blow-fill sealing machine.

9. The method of claim 1, further comprising keeping the mandrel at a temperature below 15° C.

10. A system for controlling a temperature of a biopharmaceutical product during blow-fill sealing, comprising:
a blow-fill sealing machine including a mandrel configured to introduce the biopharmaceutical product, and a mold to receive a ribbon of parison and form one or more ampoules; and
at least one cooling subunit configured and arranged to reduce a temperature of the biopharmaceutical product.

11. The system of claim 10, wherein the at least one cooling subunit includes a mandrel cooling subunit that recirculates a substance through an outer annular space of the mandrel.

12. The system of claim 10, wherein the at least one cooling subunit includes a mold cooling subunit including a standalone temperature control unit that supplies a substance to the mold.

13. The system of claim 12, wherein the substance is propylene glycol.

14. The system of claim 10, wherein the at least on cooling subunit includes one or more air knives configured to direct a stream of air onto the ribbon of parison.

15. The system of claim 14, wherein the one or more air knives includes a pair of 12″ air knives placed on opposing ends of the ribbon and configured to blow air onto the ribbon and away from the mandrel.
